(19) United States
(12) Patent Application Publication
(10) Pub. No.: US 2022/0226664 A1
(43) Pub. Date: Jul. 21, 2022

(54) Localized Voltage Generation in Volume Conductors

(71) Applicant: David Hunn, Provo, UT (US)

(72) Inventor: David Hunn, Provo, UT (US)

(21) Appl. No.: 17/576,916

(22) Filed: Jan. 15, 2022

Related U.S. Application Data
(60) Provisional application No. 63/138,446, filed on Jan. 16, 2021.

(51) Int. Cl. A61N 2/02

(52) U.S. Cl. CPC A61N 2/02

(57) ABSTRACT

A method and apparatus are disclosed for generating electrical activity in a volume conductor which has a low-frequency component that is localized within the volume conductor. This is accomplished by applying a non-localized high-frequency electromagnetic stimulus to the bulk of the volume conductor, simultaneous with the application of a high-frequency acoustic stimulus which is synchronized or partially synchronized with the electromagnetic stimulus and which is focused at a target focal region within the volume conductor.

### FIELD OF THE INVENTION

The present invention relates to the fields of acoustics, electromagnetics, and medicine. Specifically, the present invention relates to modulation and control of electrical signals within a volume conductor.

### BACKGROUND OF THE INVENTION

The electrical potentials and currents in a volume conductor in response to an external electrical or magnetic stimulus have been extensively studied and modeled, and although it is possible to optimize an external stimulus to produce deeper penetration into the bulk of a volume conductor, it is not in general possible to produce a voltage or current far from the volume conductor's surface without producing a larger voltage or current close to the surface. This poses challenges in the fields of medicine and biology, where biological tissue acts as a volume conductor. It is often desirable to electrically stimulate electrically sensitive or active biological tissues, but these sensitive or active tissues often lie deep within an organism, requiring a significant stimulus to be applied to the surface of the organism in order to reach the deep tissues. This approach has the disadvantage that the resulting electrical stimulus has very poor spatial localization and may stimulate nearby tissues, the stimulation of which is undesirable. For this reason, it is often necessary to insert electrodes into biological tissue when localized stimulation is n required, an action which is invasive and generally undesirable.

For this reason, a method of producing localized electrical stimulation of a region deep within a volume conductor is valuable to the fields of biology and medicine. Such a method may also be useful in other fields such as plasma physics, where a plasma acts as a volume conductor, or microfluidics, where control and steering of colloidal particles using electrical potentials is an active area of research.

### SUMMARY OF THE DESCRIPTION

The effect generated by the present invention is a band-limited electrical signal localized within a volume conductor. This effect is brought about by the application of a non-localized high-frequency electromagnetic stimulus to the bulk of the volume conductor, simultaneous with the application of a high-frequency acoustic stimulus which is synchronized or partially synchronized with the electromagnetic stimulus and which attains a maximum amplitude at a target focal point within the volume conductor.

### BRIEF DESCRIPTION OF THE DRAWINGS

The present invention is illustrated by way of example and not limitation in the figures of the accompanying drawings in which like references indicate similar elements.

FIG. 1 illustrates the essential elements of the present invention and their relation to one another, as well as a representation of the desired effects.

FIG. 2 illustrates application of a focused acoustic wave with displacement parallel to the direction of an electric field applied to the bulk material, according to one embodiment of the present invention.

FIG. 3 illustrates the effect of the acoustoelectric effect on the volume conductor in the focal region of the acoustic wave for the acoustic/electric oscillation shown in FIG. 2.

FIG. 4 illustrates the effect of nonuniform acoustic displacement on the volume conductor in the focal region of the acoustic wave for the acoustic/electric oscillation shown in FIG. 2.

FIG. 5 illustrates application of a focused acoustic wave with displacement perpendicular to a magnetic field applied to the bulk material, according to one embodiment of the present invention.

FIG. 6 illustrates the effect of electromagnetic induction on the volume conductor in the focal region of the acoustic wave for the acoustic/magnetic oscillation shown in FIG. 5.

FIG. 7 illustrates application of both electric and magnetic field components in conjunction with a focused acoustic wave and their synchronization to generate a localized effect, according to one embodiment of the present invention.

FIG. 8 illustrates the generation of an intermediate-frequency acoustic effect localized at the intersection of two focused, higher-frequency acoustic waves and its synchronization with applied magnetic or electric fields, according to one embodiment of the present invention.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

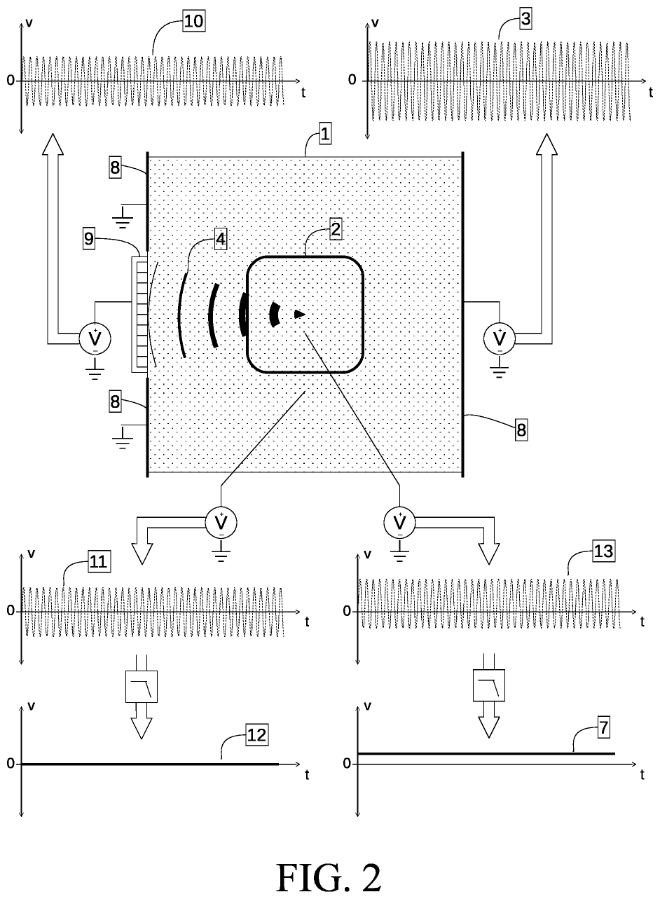

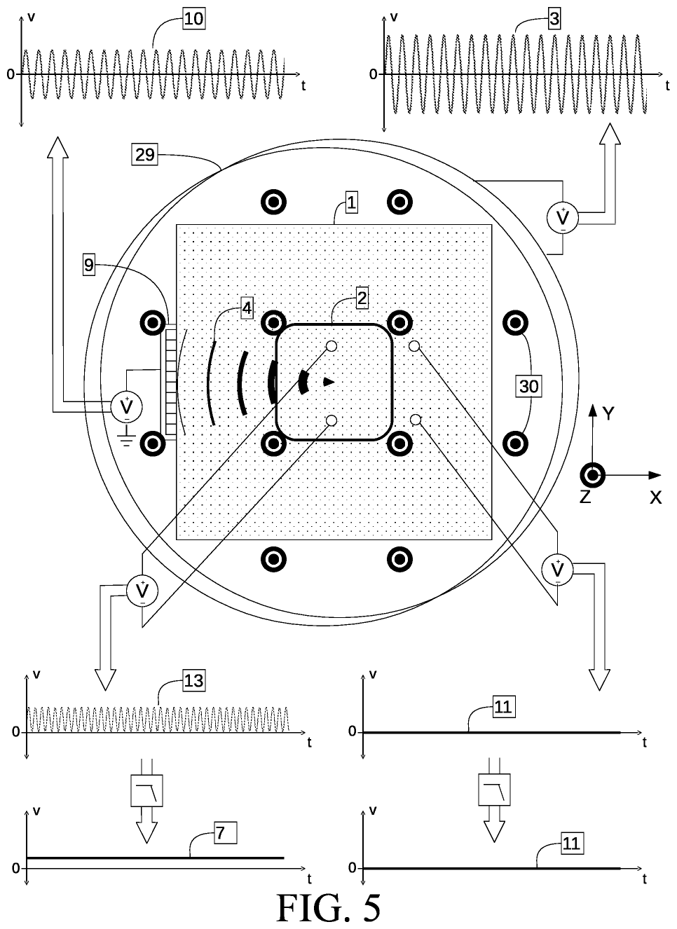

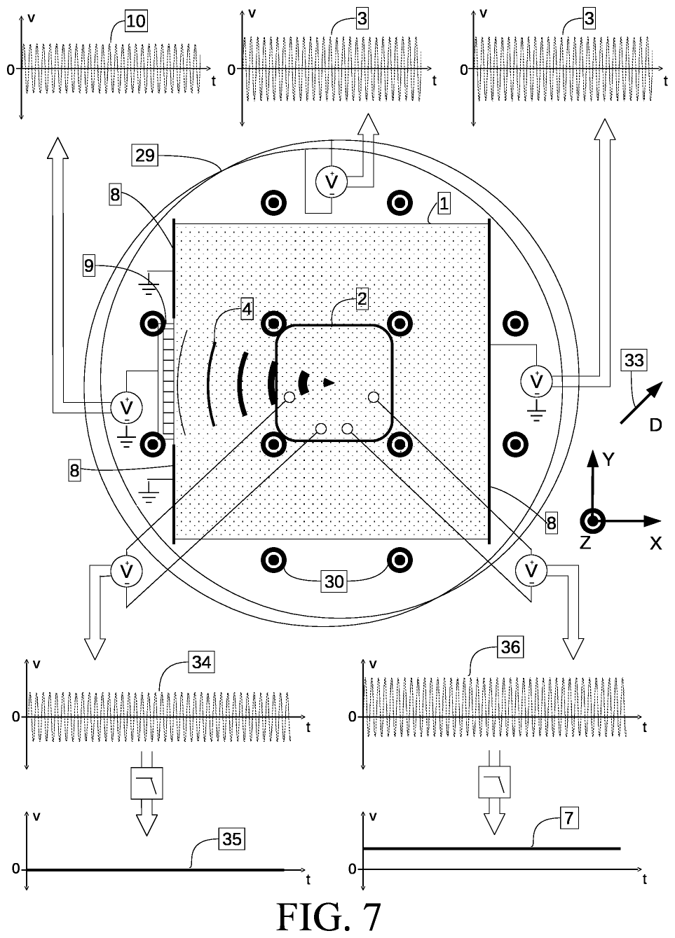

A first necessary element of the present invention is the application of an electromagnetic stimulus to the bulk of the volume conductor. This stimulus may be primarily electric, such as the application of an electric potential across the volume conductor, as shown in FIG. 2, primarily magnetic, such as the application of a magnetic field to the volume conductor, as shown in FIG. 5, or a combination of electric and magnetic, as shown in FIG. 7. The electromagnetic stimulus may be applied using any of the known application methods, including surface electrodes and field coils. The electromagnetic stimulus may be substantially uniform over the volume conductor, or it may have a generally greater amplitude in the vicinity of the target focal region, but because of the properties of volume conductors, its amplitude will inevitably be greatest near the surface of the volume conductor.

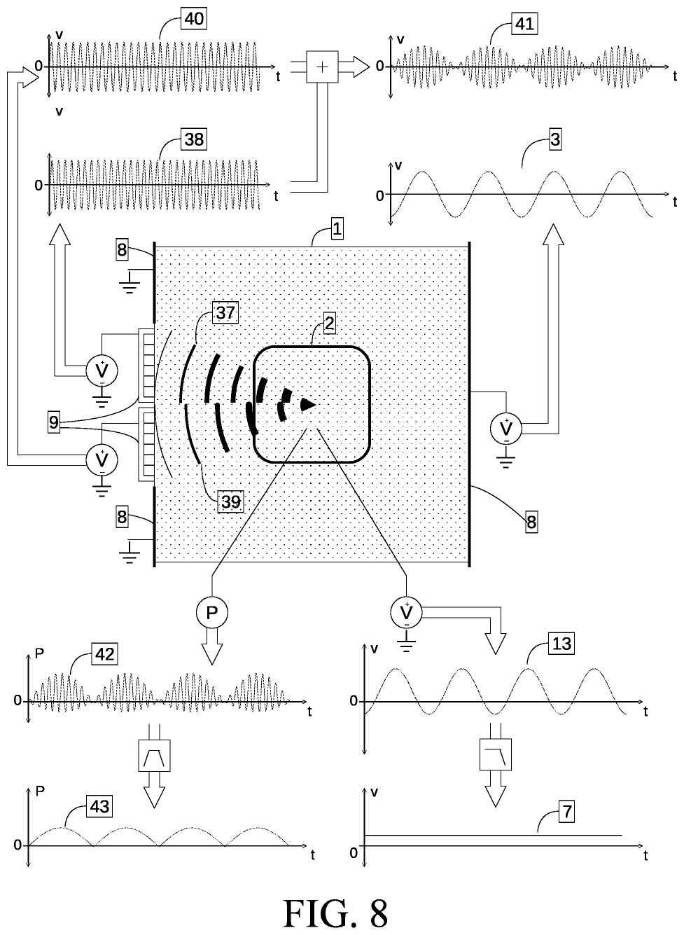

A second necessary element of the present invention is an acoustic stimulus. The acoustic stimulus may have any required frequency components, including ultrasound or infrasound. The acoustic stimulus is generated and applied to the volume conductor in such a way that the acoustic displacement, velocity, or pressure, or the component of the displacement, velocity, or pressure that synchronizes with the electromagnetic stimulus, attains a maximum amplitude in a focal region within the volume conductor. The focal region may comprise the entire volume conductor, but in most embodiments, it comprises a volume contained within the bulk of the volume conductor away from the surface of the volume conductor. The acoustic stimulus may be generated, shaped, and applied to the volume conductor using any of a number of known methods, including phased arrays, acoustic lenses, and shaped transducers. The acoustic stimulus may also be constructed such that a high-frequency primary stimulus gives rise to an intermediate-frequency secondary stimulus generated at the focal region by way of radiation pressure modulation, a phenomenon used in the known field of vibro-acoustography and illustrated in FIG. 8. Additionally, the acoustic stimulus may form a standing wave, a traveling wave, or a combination of standing and traveling waves.

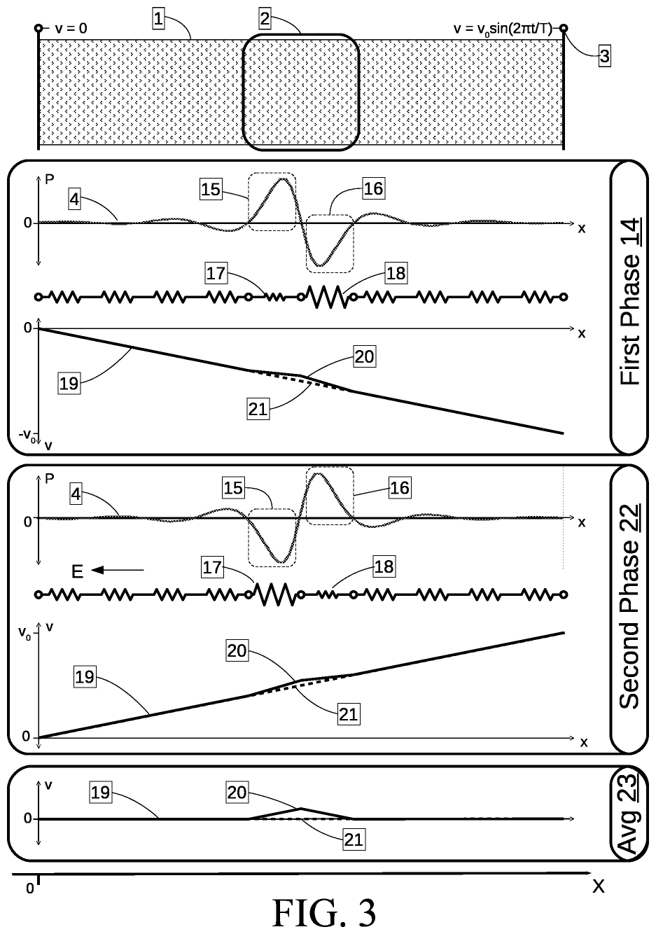

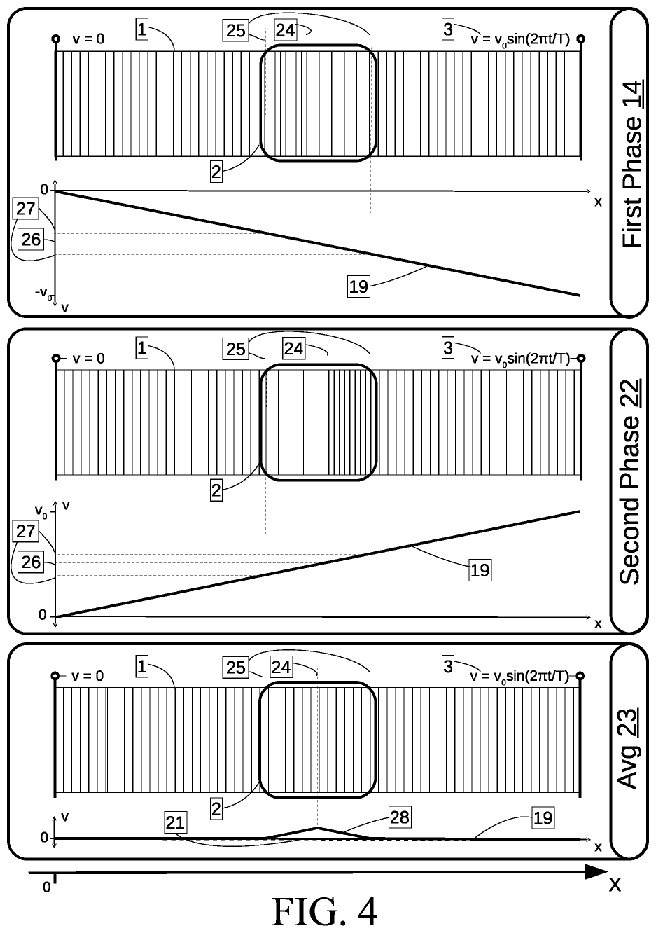

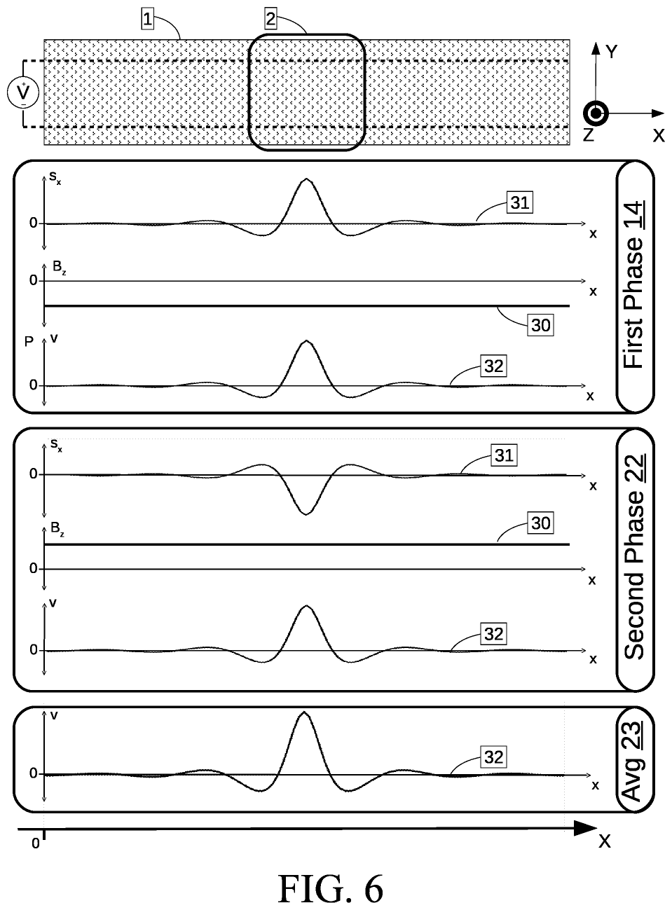

A third necessary element of the present invention is a coordination between the acoustic stimulus and the electromagnetic stimulus. The electromagnetic and acoustic stimuli must be temporally coordinated such that the acoustic activity in the target focal region is synchronized or partially synchronized with the electromagnetic stimulus in such a way that the response of the volume conductor to the electromagnetic stimulus is modulated so as to produce a lower-frequency component. This modulation may arise as a result of various effects, including electromagnetic induction on a moving portion of the volume conductor as illustrated in FIG. 6, the acoustoelectric effect on volume conductors as illustrated in FIG. 3, and nonuniform acoustic displacement of the volume conductor as illustrated in FIG. 4.

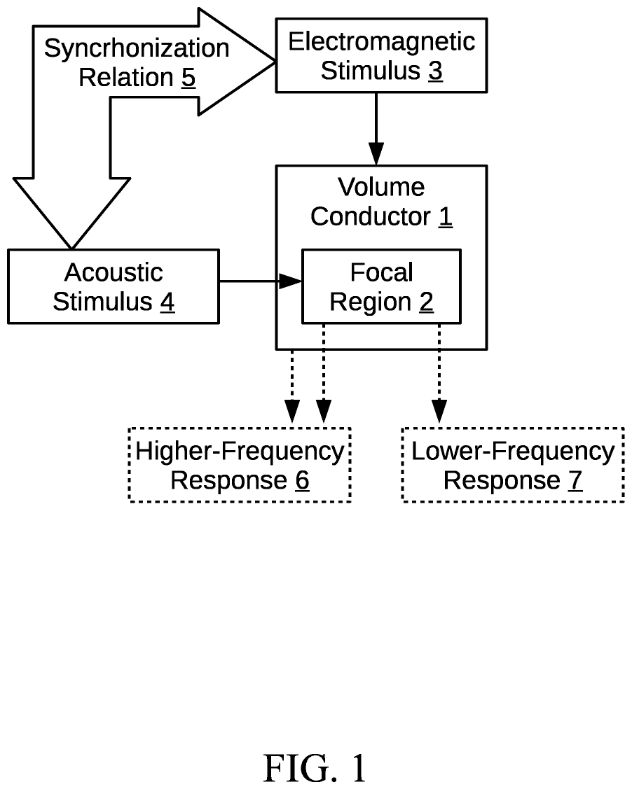

FIG. 1 illustrates the necessary components of the present invention. The volume conductor 1 contains a target focal region 2 in which a certain electrical response is desired. An electromagnetic stimulus 3 is applied to the bulk of the volume conductor 1, including the focal region 2. The acoustic stimulus 4 has a synchronization relation 5 with the electromagnetic stimulus 3 and is applied to the volume conductor 1, and particularly to the focal region 2. The electromagnetic stimulus 3 produces a higher-frequency response 6 in the bulk of the volume conductor 1, while in the focal region 2, the combination of the acoustic stimulus 4 and the electromagnetic stimulus 3 produces both the higher-frequency response 6 found throughout the volume conductor 1 and a desired lower-frequency response 7.

FIG. 2 illustrates an embodiment of the present invention in which the electromagnetic stimulus 3 is primarily electric and is applied to the volume conductor 1 via surface electrodes 8. The electromagnetic stimulus 3 in this case comprises a sinusoidal waveform with period T. The acoustic stimulus 4 is applied by a phased array ultrasound transducer 9 in contact with the volume conductor 1. In this case, the acoustic stimulus 4 comprises a sinusoidal waveform 10, also with period T, and focused on the target focal region 2. In this case, the unmodulated voltage 11 produced in the volume conductor 1 far from the focal region 2 is a simple sinusoidal waveform of period T, with a constant lower-frequency component 12 of zero. The focal region's voltage 13 consists of a sinusoidal waveform of period T with a nonzero lower-frequency component 7 consisting of a constant DC voltage. The lower-frequency component 7 is produced as a result of the combination of the acoustoelectric effect illustrated in FIG. 3 and the effect of nonuniform acoustic displacements illustrated in FIG. 4. Although the lower-frequency response 7 in this example is a DC signal, changing the synchronization relation between the electromagnetic stimulus 3 and the acoustic stimulus 4 can produce a lower-frequency component 7 with a variety of waveforms.

FIG. 3 illustrates the mechanism by which the acoustoelectric effect gives rise to a lower-frequency response 7 in the focal region 2 when a primarily electric stimulus 3 is applied in conjunction with a synchronized or partially synchronized acoustic stimulus 4. During a first phase 14 of the electric and acoustic stimuli, the pressure in the −x side 15 of the focal region 2 is elevated, while the pressure in the +x side 16 is reduced. Due to the acoustoelectric effect, this results in a reduction in the resistivity of the −x side 17 and an increase in the resistivity of the +x side 18. At the same time, in this embodiment, the electric stimulus 3 is applied with a negative potential on the +x side of the volume conductor 1. The result is an electric potential gradient 19 which decreases steadily with increasing x, except in the focal region 2, where the reduced resistivity 17 and increased resistivity 18 results in a central-region potential 20 which is somewhat elevated over the unmodulated electric potential 21. During a second phase of the electric and acoustic stimuli, the pressure in the −x side 15 of the focal region 2 is reduced, while the pressure in the +x side 16 is elevated, resulting in an increase in the resistivity of the −x side 17 and a decrease in the resistivity of the +x side 18. At the same time, in this embodiment, the electric stimulus 3 is applied with a positive potential on the +x side of the volume conductor 1. The result is an electric potential gradient 19 which increases steadily with increasing x, except in the focal region 2, where the increased resistivity 17 and decreased resistivity 18 results in a central-region potential 20 which is somewhat elevated over the unmodulated electric potential 21. The resulting average case 23 between the first phase 14 and the second phase 22 (and also, in this embodiment, the average of all phases of the electric and acoustic stimuli) has an average electrical potential 19 which is zero except in the focal region 2, where the central-region potential 20 is more positive than the zero-valued unmodulated electric potential 21.

FIG. 4 illustrates the mechanism by which nonuniform acoustic displacements give rise to a lower-frequency response 7 in the focal region 2 when a primarily electric stimulus 3 is applied in conjunction with a synchronized or partially synchronized acoustic stimulus 4. In this embodiment, during a first phase 14 of the electric and acoustic stimuli, the position 24 of the central element of the focal region 2 is displaced in the −x direction relative to the positions 25 of the elements at the edge of the focal region. At the same time, in this embodiment, the electric stimulus 3 is applied with a negative potential on the +x side of the volume conductor 1. The result is an electric potential gradient 19 which decreases steadily with increasing x (neglecting the effects of the acoustoelectric effect, illustrated in FIG. 3). Because the central element has a position 24 displaced toward the more positive potential, it takes on a potential 26 which is elevated relative to the mean of the potentials 27 at the edge of the focal region. During a second phase 22 of the electric and acoustic stimuli, the position 24 of the central element of the focal region 2 is displaced in the +x direction relative to the positions 25 of the elements at the edge of the focal region. At the same time, in this embodiment, the electric stimulus 3 is applied with a positive potential on the +x side of the volume conductor 1. The result is an electric potential gradient 19 which increases steadily with increasing x (neglecting the effects of the acoustoelectric effect, illustrated in FIG. 3). Because the central element has a position 24 displaced toward the more positive potential, it takes on a potential 26 which is elevated relative to the mean of the potentials 27 at the edge of the focal region. The resulting average case 23 between the first phase 14 and the second phase 22 (and also, in this embodiment, the average of all phases of the electric and acoustic stimuli) has an average electrical potential 19 which is zero except in the focal region 2, where the average potential 28 of the elements in this region are more positive than the zero-valued unmodulated electric potential 21.

FIG. 5 illustrates an embodiment of the present invention in which the electromagnetic stimulus 3 is primarily magnetic and is applied to the volume conductor 1 via field coils 29. The electromagnetic stimulus 3 in this case comprises a sinusoidal waveform with period T. The direction of the magnetic field 30 in this embodiment is perpendicular to the direction of displacement of the (longitudinal) acoustic stimulus 4 and is shown in the +z direction (out of page). The acoustic stimulus 4 is applied by a phased array ultrasound transducer 9 in contact with the volume conductor 1. In this case, the acoustic stimulus 4 comprises a sinusoidal waveform 10, also with period T, and focused on the target focal region 2. In this case, both the unmodulated voltage 11 and the low-pass filtered voltage 12 produced in the volume conductor 1 far from the focal region 2 is equal to a constant voltage of zero. The focal region's voltage 13 consists of a sinusoidal waveform of period T/2 having minimum voltage of zero. This waveform therefore has a nonzero lower-frequency component 7 consisting of a constant DC voltage. The lower-frequency component 7 is produced as a result of the electromagnetic induction caused by the interaction of the magnetic field 30 and the acoustic movement of the volume conductor 1, illustrated in FIG. 6. Although the lower-frequency response 7 in this example is a DC signal, changing the synchronization relation between the electromagnetic stimulus 3 and the acoustic stimulus 4 can produce a lower-frequency component 7 with a variety of waveforms.

FIG. 6 illustrates the mechanism by which electromagnetic induction gives rise to a lower-frequency response 7 in the focal region 2 when a primarily magnetic stimulus 3 is applied in conjunction with a synchronized or partially synchronized acoustic stimulus 4. In this embodiment, during a first phase 14 of the magnetic and acoustic stimuli, the velocity 31 of the elements of the volume conductor 1 in the x-direction (denoted sₓ) is strongly positive in the central area of the focal region 2. At the same time, in this embodiment, the magnetic field 30 is applied in the −z direction (into the page in this figure) throughout the volume conductor 1. As a result of the movement 31 of the central elements of the volume conductor 1 in the +x direction and the applied magnetic field 30 in the −z direction, an induced electromotive force produces a potential difference 32 in the +y direction in the central areas of the focal region 2. During a second phase 22 of the magnetic and acoustic stimuli, the velocity 31 of the elements of the volume conductor 1 in the x-direction is strongly negative in the central area of the focal region 2. At the same time, in this embodiment, the magnetic field 30 is applied in the +z direction (out of the page in this figure) throughout the volume conductor 1. As a result of the movement of the central elements of the volume conductor 1 in the −x direction and the applied magnetic field 30 in the +z direction, an induced electromotive force produces a potential difference 32 in the +y direction in the central areas of the focal region 2. The resulting average case 23 between the first phase 14 and the second phase 22 (and also, in this embodiment, the average of all phases of the electric and acoustic stimuli) has an average electrical potential difference 32 which is approximately zero except in the focal region 2, where the average potential difference is primarily positive and much larger in magnitude than the potential difference outside the focal area 2.

FIG. 7 illustrates an embodiment of the present invention in which the electromagnetic stimulus 3 comprises a mixture of electric and magnetic stimulus. In this example, the magnetic field 30 is applied to the volume conductor 1 via field coils 29 with a direction perpendicular to the direction of displacement of the (longitudinal) acoustic stimulus. The electric field portion of the electromagnetic stimulus 3 is applied to the volume conductor 1 via surface electrodes 8 in a direction parallel to the direction of the acoustic stimulus 4. Both the electric and magnetic portions of the electromagnetic stimulus 3 comprise sinusoidal waveforms with period T. In this figure, the electric field and the acoustic stimulus are directed in the x direction, and the magnetic field is directed in the z direction (out of page). The acoustic stimulus 4 is applied by a phased array ultrasound transducer 9 in contact with the volume conductor 1. In this case, the acoustic stimulus 4 comprises a sinusoidal waveform 10, also with period T, and focused on the target focal region 2. In this case, the lower-frequency component 7 of the voltage response in the focal region 2 is directed in a desired direction 33 midway between the +x and +y directions, at 45 degrees relative to the +y direction and 45 degrees relative to the +x direction. Thus, the unmodulated voltage 34 measured perpendicular to this direction (in the direction midway between the −x and +y directions) consists of a sinusoidal waveform, and its lower-frequency component 35 is zero. On the other hand, the unmodulated voltage 36 measured in the desired direction 33 consists of a sinusoidal waveform with a positive DC offset, so the desired lower-frequency component 7 is positive. Although the lower-frequency response 7 in this example is a DC signal in a fixed direction, changing the synchronization relation between the electric component of the electromagnetic stimulus 3, the magnetic component of the electromagnetic stimulus 3, and the acoustic stimulus 4 can produce a lower-frequency component 7 which varies arbitrarily in both magnitude and direction.

FIG. 8 illustrates an embodiment of the present invention in which the electromagnetic stimulus 3 is primarily electric and is applied to the volume conductor 1 via surface electrodes 8. The electromagnetic stimulus 3 in this case comprises a sinusoidal waveform with frequency fₑ. The acoustic stimulus 4 is applied by a pair of phased array ultrasound transducers 9 in contact with the volume conductor 1. In this case, the acoustic stimulus 4 is composed of two separate acoustic waves, one 37 with a higher acoustic frequency generated by a sinusoidal waveform 38 with frequency fₕ, and one 39 with a lower acoustic frequency generated by a sinusoidal waveform 40 with frequency fₗ such that fₕ−fₗ=fₑ/2. By this means, at the intersection of the acoustic waves 37 and 39 in the focal region 2, a summed acoustic waveform 41 is produced which has a base frequency (fₕ+fₗ)/2 and a modulation frequency of fₑ. Due to acoustic radiation pressure, the net pressure 42 in the focal region 2 is highest at the peaks of the modulated signal, producing an intermediate-frequency acoustic pressure component 43 with frequency fₑ.

In this case, the focal region's voltage 13 consists of a sinusoidal waveform of frequency fₑ with a nonzero lower-frequency component 7 consisting of a constant DC voltage. The lower-frequency component 7 is produced as a result of the intermediate-frequency acoustic radiation waveform 43 interacting with the electromagnetic stimulus 3 via a combination of the acoustoelectric effect illustrated in FIG. 3 and the effect of nonuniform acoustic displacements illustrated in FIG. 4. Although the lower-frequency response 7 in this example is a DC signal, changing the synchronization relation between the electromagnetic stimulus 3 and the acoustic stimulus 4 can produce a lower-frequency component 7 with a variety of waveforms.

1. A system comprising:
a first component suited to apply an electromagnetic stimulus to all or part of a volume conductor,
a second component suited to apply an acoustic stimulus to all or part of the target volume conductor such that at least one component of the acoustic activity produced in the volume conductor is concentrated in a target focal region of the volume conductor,
an acoustic stimulus applied to the volume conductor via the second component so as to produce acoustic activity within the volume conductor, with at least one component of the produced acoustic activity being concentrated in the target focal region,
an electromagnetic stimulus applied to the volume conductor via the first component,
a synchronization relation between the electromagnetic stimulus and the produced acoustic activity such that the resulting electrical activity produced within the volume conductor includes a desired component which is of lower frequency than the produced acoustic activity or the electromagnetic stimulus and which is concentrated in the target focal region.

2. The invention of claim 1 in which the electromagnetic stimulus is entirely or primarily electrical.

3. The invention of claim 2 in which the desired lower-frequency component of the produced electrical activity results entirely or partially from the interaction between the change in resistivity of the volume conductor caused by the acoustic activity and the electric current caused by the electrical stimulus.

4. The invention of claim 3 in which the desired lower-frequency component of the produced electrical activity is a voltage gradient produced due to a portion of the target focal region alternating between a lower-resistivity state and a higher-resistivity state in synchronization with the electric current in at least a portion of the volume conductor alternating between a positive and a negative direction.

5. The invention of claim 2 in which the desired lower-frequency component of the produced electrical activity results entirely or partially from the interaction between the physical displacement of a portion or portions of the volume conductor caused by the acoustic activity and the electrical potential gradient caused by the electrical stimulus.

6. The invention of claim 5 in which the desired lower-frequency component of the produced electrical activity is a voltage gradient produced due to a portion of the target focal region alternating between a state of higher and lower physical and electrical admittive proximity to volume conductor regions in a positive direction in synchronization with the electrical potential gradient in at least a portion of the volume conductor alternating between a positive and a negative direction.

7. The invention of claim 1 in which the electromagnetic stimulus is entirely or primarily magnetic.

8. The invention of claim 7 in which the desired lower-frequency component of the produced electrical activity results entirely or partially from the interaction between the velocity of a portion or portions of the volume conductor caused by the acoustic activity and the magnetic field caused by the magnetic stimulus.

9. The invention of claim 8 in which the desired lower-frequency component of the produced electrical activity is a voltage gradient produced via electromagnetic induction due to a portion of the target focal region alternating between motion in a positive direction and motion in a negative direction in synchronization with the magnetic field in the volume conductor alternating between a positive direction perpendicular to the directions of motion and a negative direction perpendicular to the directions of motion.

10. The invention of claim 1 in which the electromagnetic stimulus comprises both an electrical and a magnetic component.

11. The invention of claim 10 in which the desired lower-frequency component of the produced electrical activity results from a combination of
the interaction between the velocity of a portion or portions of the volume conductor caused by the acoustic activity and the magnetic field caused by the magnetic stimulus,
the interaction between the physical displacement of a portion or portions of the volume conductor caused by the acoustic activity and the electrical potential gradient caused by the electrical stimulus, and
the interaction between the velocity of a portion or portions of the volume conductor caused by the acoustic activity and the magnetic field caused by the magnetic stimulus.

12. The invention of claim 1 in which the produced acoustic activity includes a primary acoustic activity produced directly by the acoustic stimulus and a secondary acoustic activity generated at a lower frequency than that of the primary acoustic activity due to the effect of time-varying radiation pressure produced by the primary acoustic activity, where the electromagnetic stimulus has a synchronization to the secondary acoustic activity.
